(19) United States
(12) Patent Application Publication
(10) Pub. No.: US 2005/0283110 A1
(43) Pub. Date: Dec. 22, 2005

(54) Ultrasound-mediated drug delivery

(75) Inventors: Anthony Atala, Winston Salem, NC (US); Marcelle Machlouf, Brookline, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: CHILDREN'S MEDICAL CENTER CORPORATION, Boston, MA (US)

(21) Appl. No.: 11/179,050

(22) Filed: Jul. 11, 2005

Related U.S. Application Data
(63) Continuation of application No. 09/114,810, filed on Jul. 13, 1998, now abandoned.
(60) Provisional application No. 60/074,231, filed on Feb. 10, 1998.

(51) Int. Cl. A61N 1/30

(52) U.S. Cl. 604/20

(57) ABSTRACT

Methods and apparatus are disclosed for treating physiological problems, and for providing rapid, efficacious transdermal treatment, for example, of muscle sprains, erectile dysfunction, or baldness, without requiring the use of needles or other invasive interventions. A topical therapeutic agent and ultrasound energy are applied to a tissue surface, e.g., the skin, such that the ultrasound enhances transdermal penetration of the agent. The invention is especially useful in localized delivery of a controlled dosage of a therapeutic agent to the small blood vessels and capillaries beneath the skin's surface.

### REFERENCE TO RELATED APPLICATION

The present application claims the benefit of, and incorporates by reference, the commonly owned, co-pending U.S. provisional application No. 60/074,231, filed Feb. 10, 1998.

### BACKGROUND OF THE INVENTION

The technical field of this invention is medical treatments and devices. In particular, medical methods and devices are disclosed for enhancing transcutaneous absorption of drugs. The invention is useful for a variety of purposes, including the treatment of muscle sprains and inflammation, treatment of male erectile dysfunction, treatment of hereditary baldness and other applications.

Muscle sprains typically occur when over-exercise or a traumatic event causes a joint to move beyond its normal range if motion and tissues of the muscle's tendons or ligaments are torn or stretched. The results of such sprains are typically rapid swelling, tenderness or pain, and/or impaired joint function. The treatment of muscle sprains usually involves applying ice to the affected region, rest and aspirin or other analgesic agents. Only in serious cases are stronger anti-inflammatory drugs recommended because they must be applied systemically (e.g., orally) or by injection. The use of topical analgesics and/or topical anti-inflammatory agents is considered of marginal effectiveness, despite the plethora of products sold as over-the-counter pain relief agents. Topical agents are largely unable to pass transdermally to the capillary beds that surround the afflicted tissue and, hence, usually can not delivery an sufficient dosage of the medication to the site of injury.

Erectile dysfunction is a common medical problem among men. The incidence increases with age and other medical disorders such as diabetes mellitus. The dysfunction is characterized by an inability to obtain a penile erection or to sustain an erection. Several vasoactive drugs have been tried for the clinical evaluation and treatment of impotence. Among them, papaverine and prostaglandin E1 (PGE 1) are the most widely used. These agents are strong smooth muscle relaxants, which can induce penile erection after intracorporal injection (intracavernosal therapy). However, intracavemosal injections have several side effects. Repeated injections can cause fibrosis of the penile shaft and priapism (prolonged erection). Moreover, this therapy has associated disadvantages such as discomfort, pain, and injection anxiety, which can result in patient rejection of the therapy and/or a less than optimal level of satisfaction even when erection is achieved.

Recently, a new class of drugs have been greeted with considerable enthusiasm. These drugs, known as phosphodiesterase type 5 inhibitors, block an phosphodiesterase enzyme that functions to break down the chemical GMP, produced during sexual stimulation. By ensuring that GMP remains in the penile tissue and surrounding blood vessels, these inhibitors help initiate and maintain an erection. The first of this new class of drugs, sildenafil (sold by Pfizer under the trademark Viagra™) has already become widely prescribed. Unfortunately, the drug must be taken systemically and many subjects have reported side effects such as headaches, nausea and facial flushing and indigestion.

The transdermal application of vasoactive drugs for treatment of erectile dysfunction has been studied in vitro and in vivo. While transdermal application avoids certain of the disadvantages of injection, transdermal therapies to date have been associated with limited success. The side effects of sildenafil and other enzyme inhibitors, as well as, systemic vasoactive drugs could be diminished significantly, if a suitable transdermal delivery mechanism was available because the necessary doses could be lowers and the drugs would be less likely to affect remote body organs.

Hereditary baldness usually occurs in men and is characterized by a gradual thinning of the hair, generally, and a receding hairline. Over time the recession continues until the individual is left with a horseshoe like pattern of hair around the sides of the head. Recently, the drug minoxidil (originally developed to treat high blood pressure) has been found to inhibit hairline recession and, in some cases, stimulate new hair growth. The drug is now sold as a over-the-counter product, usually in the form of a topical lotion. Although the mechanism of action is not fully understood, it appears that the drug stimulates the activity of dormant hair papilla, causing the hair roots to grow a new shafts through their follicles. Again, the effectiveness of this therapy is limited by the ability to topically deliver sufficient amounts of the drug to the papilla and surrounding beds of blood vessels.

There exists a need to better transdermal drug delivery systems, for the treatment of physiological problems generally, and for the treatment of muscle sprains, erectile dysfunctions, and baldness, in particular. Systems and methods that can improve the effectiveness of existing agents and/or permit systemic agents to be delivered locally would satisfy a long-felt need in the art.

### SUMMARY OF THE INVENTION

Methods and apparatus are disclosed for treating physiological problems, and for providing rapid, efficacious transdermal treatment of treatment of muscle sprains, erectile dysfunctions, and baldness, in particular, without requiring the use of needles or other invasive interventions. The present invention applies both a topical therapeutic agent and ultrasound energy to a tissue surface, such as the skin, such the ultrasound enhances transdermal penetration of the agent. The invention is especially useful in localized delivery of a controlled dosage of a therapeutic agent to the small blood vessels and capillaries beneath the skin's surface.

In one aspect, the invention provides a method for treatment of muscle sprains and other superficial tissue inflammations. The method includes the steps of contacting the skin surface of a subject in need of such treatment with an effective amount of an agent capable of treating the inflammation, and applying ultrasound energy to the skin surface, such that ultrasound-mediated drug absorption occurs and the inflammation is treated. Agents useful in treatment of muscle sprains include analgesics, anti-inflammatory agents, cortisone derivatives and other steroids.

In another aspect, the invention provides a method for treatment of erectile dysfunction. The method includes the steps of contacting the skin surface of a subject in need of such treatment with an effective amount of an agent capable of treating erectile dysfunction, and applying ultrasound energy to the skin surface, such that ultrasound-mediated drug absorption occurs and the erectile dysfunction is treated. Agents capable of treating erectile dysfunction include recently developed enzyme inhibitors, such as sildenafil or alprostadil, as well as more traditional vasoactive agents, such as papaverine, minoxidil, prostaglandins, organic nitrites, inhibitors of the renin-angiotensin system, and inducible Nitric Oxide Synthase (iNOS) agents.

In yet another aspect, the invention provides a method for treatment or prevention of hair loss. The method includes the steps of contacting the skin surface of a subject in need of such treatment with an effective amount of an agent capable of inducing hair growth, and applying ultrasound energy to the skin surface, such that ultrasound-mediated drug absorption occurs and the hair papilla are stimulated. Agents useful in controlling hair loss include minoxidil, finasteride, fabao-101, cyproterone acetate, ethinyl estradiol, aldactone and spironolactone. More generally, the invention is useful for treatment of hair loss with anti-androgen therapies (e.g., localized delivery of therapeutic agents designed to block the enzymatic conversion of testosterone into dihydrotestosterone(DHT)) and/or vasodialator therapies design to increase blood flow to the follicle roots.

Transdermal administration of drugs, such as cortisone derivatives, sildenafil, papaverine and minoxidil (among others) can offer a safe, non-painful alternative to previously known therapies. However, conventional transdermal application of such drugs has met with reduced success, possibly due to the stratum corneum layer of the skin, which is resistant to drug penetration through the skin to the underlying tissues such as torn ligaments, tendons, muscle tissue, the corpus cavernosum of a penis, and/or the papillae and surrounding structures of hair follicles.

In one aspect of the apparatus, the invention provides a device for the treatment of muscle sprains and the like. The device include means for applying an effective amount of an agent capable of capable of treating the muscle aliment to a tissue surface of a subject, and ultrasound means, operatively coupled to the means for applying the drug, for promoting transdermal absorption of the drug through the tissue of the subject. In one embodiment, the device has the form of a hand held instrument. The means for applying a drug can be a reservoir which contains the drug, and, optionally, a physiologically-acceptable carrier or excipient. The reservoir can be of the type employed in conventional transdermal patch applications or it can one specially designed to be integrated with the hand-held ultrasound applicator. The applicator can also include a slot, reservoir, or space for receiving a (preferably replaceable) source of the therapeutic agent, and a compliant skin contacting surface.

In another aspect of the apparatus, the invention provides a device for the treatment of erectile dysfunction. The device include means for applying an effective amount of an agent capable of capable of treating erectile dysfunction to a tissue surface of a subject, and ultrasound means, operatively coupled to the means for applying the drug, for promoting transdermal absorption of the drug through the tissue of the subject. In a preferred embodiment, the device has the form of a ring or torus adapted for fitting to the penis of the subject. The means for applying a drug can again be a reservoir which contains the drug, and, optionally, a physiologically-acceptable carrier or excipient; the reservoir can be of the type employed in conventional transdermal patch applications. The device can also include a slot, reservoir, or space for receiving a (preferably replaceable) source of the drug. For example, the device can include a well for receiving a portion of a drug formulation, such as a cream or ointment, or a slot for receiving a disposable reservoir, such as a patch, which can be inserted before the device is used, and then removed and, optionally, discarded after use.

For erectile dysfunction treatment, the device is preferably small in size, such that it can be unobtrusively placed over the flaccid penis. After penile erection is achieved through use of the device, the device can be removed. However, in one embodiment, the device can be small enough such that it will not interfere with sexual intercourse and need not be removed. In certain embodiments, the ultrasound source is powered by batteries carried within the body of the device. The batteries can be of the type used for powering microelectronic devices, such as hearing aids, thereby preserving the small size of the device.

The invention also provides systems for applying an effective amount of an agent capable of capable of treating erectile dysfunction to the skin of the penis and means for applying ultrasound energy to the same skin region. The drug dispenser can comprise a condom having a coating of an active agent on an interior surface; a patch containing the active agent, analogous to conventional transdermal patches for the delivery of drugs such as scopolamine and nicotine; an applicator adapted for placement on or over the penis of the subject, having a surface impregnated with an active agent, or a reservoir containing the active agent. The applicator can further include means for dispensing the agent, preferably at a controlled rate, to the subject's skin; and other like means for delivering the drug to the subject.

In one illustrated embodiment, the means for applying the active agent includes a penile device which is placed around the penis to deliver the drug and the required ultrasound energy for appropriate drug delivery is applied with a hand held applicator. For example, a drug permeated transdermal patch can be placed in direct contact with the penile skin, and an ultrasound-producing device is placed over the patch. After a suitable time period, e.g., a few minutes, the ultrasound applicator and the patch are removed, exposing the erect phallus.

The devices and systems of the invention can also be provided with means for determining the erectile state of the penis, e.g., for detecting detumescence or loss of erection. Such means include a pressure-sensitive mechanical switch such as described previously, or an imaging ultrasound receiver for determining blood flow or penile erection. In the event of detumescence, ultrasound energy can be applied to promote renewed penile erection.

According to the methods of invention, an agent capable of treating erectile dysfunction, e.g., an agent capable of promoting penile erection, such as a a smooth muscle relaxant, is applied to a tissue surface, e.g., the skin of the patient, preferably on the penis itself, e.g., the glans or, more preferably, the shaft of the penis (or both the glans and the shaft, e.g., as when a condom containing the active agent is used). It will be appreciated, however, that the agent can be applied in other ways, e.g., intra-urethrally. In certain embodiments, the active agent can be applied to other skin surfaces, e.g., any skin surface, which will permit an effective amount of the drug to penetrate the skin and reach the penile tissues such that penile erection is promoted. An active agent can also be applied to any skin surfaces in addition to than the penis, if desired (e.g., to provide greater skin surface area for drug penetration). The agent can be any compound which can be applied to a tissue surface of the subject and which can promote penile erection. The drug agent can be applied to the skin by topical administration, e.g., by applying a cream, lotion, gel or other formulation which includes an active agent, to the skin before or during the application of ultrasound to the affected area. In certain embodiments, the drug can be applied by contracting the skin with a device which includes a coating or layer of the drug disposed thereon such that the drug is applied to the skin. A band, sheath, condom, patch, or other carrier having a skin-contacting surface can be coated with an active agent, such that the drug is applied to the skin of the user.

For example, in one embodiment, the subject administers the drug by rolling a condom onto the flaccid penis; the condom is coated on at least a portion of the interior surface thereof with an active agent (which can be formulated in a base including conventional lubricants, emollients, or other ingredients known in the art). The condom is worn for a period of time sufficient to ensure adequate application of the active agent to the skin of the subject. The condom can then be removed and ultrasound energy applied to the penis of the user; alternatively, in certain embodiments, the ultrasound energy can be provided to the penis while the condom is still worn, e.g., by application through the condom to the penis. In either case, application of ultrasound energy promotes penile erection by increasing the bioavailability of the active agent to the underlying tissues of the penis, such as the corpus cavernosum. In embodiments in which a condom is employed, the condom can be removed before sexual intercourse or alternatively can be worn during intercourse.

Therapeutic agents useful for treatment of erectile dysfunction include, without limitation, sildenafil, alprostadil, papaverine, minoxidil, prostaglandins, such as prostaglandin E2 (see, e.g., U.S. Pat. No. 5,708,031 for formulations of prostaglandins useful for topical application to the penis), organic nitrites (see, e.g., U.S. Pat. No. 5,646,181 for useful organic nitrites and formulations thereof), inhibitors of the renin-angiotensin system (see, e.g., U.S. Pat. No. 5,658,936), and/or inducible Nitric Oxide Synthase (iNOS) agents (see, e.g., U.S. Pat. No. 5,594,032) or combinations of such compounds as well as other compounds known to those of ordinary skill in the art. (The teachings of each of the cited patents are incorporated herein by reference.) Mixtures of active agents can also be employed.

In yet another aspect of the apparatus, the invention provides a device for the treatment of hair loss. The device can include mean for applying an effective amount of an agent capable of capable of treating the hair loss to the scalp, or other affected region of a subject, and ultrasound means, operatively coupled to the means for applying the drug, for promoting transdermal absorption of the drug through the tissue of the subject. In one embodiment, the device has the form of a hand held instrument. The means for applying a drug can be a reservoir which contains the drug, and, optionally, a physiologically-acceptable carrier or excipient. The reservoir can be of the type employed in conventional transdermal patch applications or it can one specially designed to be integrated with the hand-held ultrasound applicator. The reservoir can take the form of a scalp cap which is fitted over the affected area. The applicator can also include a slot, reservoir, or space for receiving a (preferably replaceable) source of the therapeutic agent. Alternatively the agent can be applied manually (e.g., massaged into the scalp).

The therapeutic agent for treatment of hair loss can be, without limitation, compounds such as minoxidil, finasteride, fabao-101, cyproterone acetate, ethinyl estradiol, aldactone and spironolactone. More generally, the agent can be an anti-androgen therapeutic agent (designed to block the enzymatic conversion of testosterone into dihydrotestosterone(DHT)) and/or a vasodilator designed to increase blood flow to the penile blood vessels.

The methods and systems of the invention can also include additional means for enhancing transdermal penetration or absorption of an active agent. For example, enhancers of transdermal drug absorption have been used to increase the efficacy of other transdermal drug administration modalities, such as transdermal skin patches and the like. Permeation enhancers which can be useful in the present invention include dimethylsulfoxide related compounds (DMSOs), 1,3-dioxacycloalkanes (SEPAs), amphoteric cations and anions, fatty acids (and their esters), fatty alcohols (and their ethers), glycols, alcohols, acetones, ketones and other organic solvents, as well as other known permeation enhancers that increase the rate or amount of active agent transported across the dermal barrier.

The ultrasound applicator preferably comprises a power source (such as a battery or transformed household current) and at least one ultrasound transducer capable of providing ultrasound energy at a frequency of between 20 kHz and 5 MHz and a power of about 0.02 to about 3 watts/cm². (Ultrasound sources are described in more detail infra). The ultrasound applicator can further include suitable control circuitry for generating complex waveforms, amplitude variations, frequency variations, constructive interference and other functional controls on the ultrasonic therapy.

The devices and systems are also preferably provided with means for switching the ultrasound source on and off at appropriate intervals, e.g., for switching on the ultrasound source to promote penile erection, after which the ultrasound source is switched off, e.g., to preserve battery life. The means can be a mechanical switch which the user can actuate at the requisite intervals, or, more alternatively, can be a timer or microprocessor-controller switch which is set or programmed before use to provide the appropriate power levels and duration. In a further embodiment, the switch can be mechanically actuated, e.g., in response to tumescence of the penis, to thereby discontinue application of the ultrasound energy when the penis has become erect. In this embodiment, the switch can include a pressure-sensitive switch member proximal to a surface of the device which contacts the penis when penile erection is achieved. Thus, upon erection of the penis, the switch is actuated and the ultrasound source is switched off. In addition to preserving battery life, by switching off the ultrasound source, excessive stimulation of erection is avoided, thereby avoiding priapism or other conditions which could cause user discomfort.

Thus, the present invention provides a more effective treatment particularly adapted to pathological or physiological conditions where it is desirable to apply a drug to a local region below but in close proximity to the stratum corneum layer of the skin. The invention allows drugs to diffuse quickly, and to be rapidly taken up by the subsurface localized blood capillary networks, while at the same time does not inactivate the drug molecules, damage healthy epidermis, cause pain or have toxicologic side effects.

It has also been discovered that ultrasound frequencies ranging between about 500 kHz and about 3 MHz can promote the therapeutic effect of topically applied agents. Although the mechanical action is not yet clear, and without intending to be bound by any particular theory, ultrasound effects are often classified into two major categories; thermal and non thermal. Cavitation is one of the major non thermal effects, in which microscopic air pockets are created and oscillated within the tissue upon exposure to the acoustic field. The energy produced by this phenomenon is partially (10-15%) radiated as an acoustic field, whereas most of it is transformed to heat, shock wave or hydrodynamic shear fields, partially disrupting biological tissue.

The occurrence of cavitation depends mainly upon ultrasound frequencies and intensities. By appropriate control of the cavitation phenomenon, significantly higher doses of therapeutic agents can be transported across the stratum corneum. In addition, induced cavitation can serve a two-fold purpose in the administration of minoxidil and other hair loss treatment agents by not only enhancing skin penetration of the agent but also by opening the hair follicles and driving the therapeutic agent into contact with the papilla through the lumens of the follicles.

For example, a male human subject can use a device of the invention, including an adjustable band or strap and an ultrasound source with a drug reservoir, as follows. Prior to sexual intercourse, the user places an effective amount of the drug, e.g., in the form of a patch containing a therapeutic agent, into a slot in the housing of the ultrasound source. The devices is then secured to the user's flaccid penis by placing the adjustable band around the shaft of the penis such that the drug patch is in contact with the skin of the penis. The user then presses a button to activate the ultrasound source; the device includes a timer which automatically provides ultrasound energy for two minutes, at a frequency of about 20 kHz and a power of about 0.2 W/cm²; and in a duty cycle of about 20%. the drug is effective to promote an erection after application of the ultrasound energy. The device can then be removed from the penis. If the user should experience loss of erection, the device can be used again to promote another erection. If necessary, a fresh drug patch can be provided to ensure that an effective amount of drug is used.

Accordingly, novel therapies have been discovered in which ultrasound waves, of different frequencies and/or intensities, are used to enhance transdermal delivery of pharmaceutic agents. Ultrasound-induced cavitation provides a non painful method for enhancing drug delivery with few or no side effects.

Most generally, the invention provides methods for transdermal therapy that include the step of contacting a tissue surface (e.g., the skin surface) of a subject in need of such treatment with an effective amount of an agent capable of capable of treating the physiological condition, and the step of applying ultrasound energy to the tissue surface, such that the condition is treated (e.g., by ultrasound-mediated drug absorption). The term “subject,” as used herein, includes mammals, including humans as well as non-human animals such as cats, dogs, horses, pigs, goats, sheep, and non-human primates. In certain embodiments, the step of contacting can include applying the active agent to the tissue surface with a patch or a sheath or condom coated with or otherwise impregnated with the active agent. In other embodiments, the agent can be applied with other devices or systems, e.g., as described herein. The tissue surface is preferably a skin surface, e.g., the skin surrounding a torn muscle, the skin of the penis, or the scalp. Other tissue surfaces, such as the interior of the urethral or vaginal openings, the mouth, and ears can also be contacted with an active agent and treated according to the methods of the invention.

In a further aspect, the invention provides devices, systems and kits for the treatment of physiological problems. The devices include an ultrasound transducer for promoting transdermal absorption of the drug through the skin of the subject. In one embodiment, the device has the form of a hand held sonicator. In another embodiment, the device can take the form of a belt-like structure adapted to fit around a body limb or the penis or the head of the subject. In yet another embodiment the system (or applicator component) can take a sheath-like or scalp-cap shape.

The applicator means for applying a drug can be a reservoir which contains the drug, and, optionally, a physiologically-acceptable carrier or excipient, or a transdermal penetration enhancer. The reservoir can be fluidically coupled via a flow regulator, to the treatment site. Alternatively, the applicator can be constructed like a conventional transdermal patch applications or it can be specifically designed to fit within a hand held housing. In the case of penile therapies the reservoir can be take the form of a condom. In the case of hair loss therapies the reservoir can be take the form of a scalp or skull cap. In other applications, drug can simply be applied to the tissue as a gel or lotion, or the ultrasound transducer can include an applicator for applying a drug to a skin surface of a subject.

The ultrasound means preferably comprises a power source (such as a replaceable or rechargeable battery, or a transformer for utilization of household current) and at least one ultrasound transducer capable of providing ultrasound energy at a frequency of between about 20 kHz and about 3 MHz. In some applications it can be desirable to modulate the frequency using a preprogrammed tuner to alter the frequency over time to enhance microcavitation effects (and other mechanisms of transdermal transport) through the skin. Similarly, the use of two or more transducers (or wave reflectors) can be desirable to induce constructive interference patterns that likewise enhance the skin-penetration rates.

The ultrasound source can be of any suitable design, e.g., a design such as certain ultrasound sources known in art. For example, ultrasound probes used in Doppler sonography are well known, and, by generating appropriate ultrasound frequencies and energies as described herein, can be used in the methods, devices, and systems of the present invention.

The ultrasound source is preferably connected to a power supply and to suitable control means for regulating the ultrasound signal produced by the ultrasound source. The power supply and control means (e.g., circuitry) can be disposed within or external to a body-contacting portion of a device or system of the invention, and is operatively connected to the ultrasound source, e.g., by means of wires running from the ultrasound source, to the power supply and control means. The control means can be provided with a user interface for setting parameters such as the power, duty cycle, and pulse duration of the ultrasound energy provided by the ultrasound source. Suitable control means will apparent to one of ordinary skill in the art. It will be appreciated that an ultrasound transducer such as a piezoelectric crystal can also be operated as a receiver for ultrasound waves reflected from tissue surrounding the transducer. Thus, the control means can be provided with appropriate receiver circuitry to provide monitoring capability to the systems of the invention. Such monitoring capability can be used by the systems or devices of the invention in a feedback control loop, e.g., for determining when penile erection has been achieved or blood flow to the penis has been enhanced.

### BRIEF DESCRIPTION OF THE DRAWINGS

The foregoing and other aspects of the present invention will be appreciated with reference to the detailed description of the invention which follows, which read in conjunction with the accompanying drawings wherein:

FIG. 1 is a schematic, perspective illustration of an ultrasound applicator for mediating drug delivery according to the invention;

FIG. 2 is a partially cross-sectional side view of the transducer head of the apparatus of FIG. 1;

FIG. 3 is a schematic, perspective view of a drug dosage dispenser for use in conjunction with the transducer head assembly of FIG. 2;

FIG. 4 is a schematic, perspective illustration of an alternative embodiment of an ultrasound-mediated drug delivery apparatus according to the invention;

FIG. 5 is a schematic, cross-sectional front view of the apparatus of FIG. 4;

FIG. 6 is a top view of the drug receptacle assembly of the apparatus of FIG. 4;

FIG. 7 is a schematic illustration of a dosage dispenser for use in connection with the apparatus of FIG. 4;

FIG. 8 is a schematic side view of an ultrasound applicator coupled to the apparatus to FIG. 4;

FIG. 9 is another alternative embodiment of the invention, employing a belt and integrated ultrasound transducer arrangement;

FIG. 10 is another alternative embodiment of the invention, employing a condom-like sheath and a toroidal ultrasound transducer arrangement;

FIG. 11 is a cross-sectional top view of the apparatus of FIG. 10;

FIG. 11A is a schematic illustration of constructive interference patterns treated by multiple transducers disposed in the toroidal arrangement of FIG. 11;

FIG. 12 is a schematic block diagram of an ultrasound transducer control system according to the invention; and

FIG. 13 is a graph of amplitude versus time, illustrating a variable frequency protocol for the application of ultrasound to enhance transdermal penetration, according to the invention.

### DETAILED DESCRIPTION OF THE INVENTION

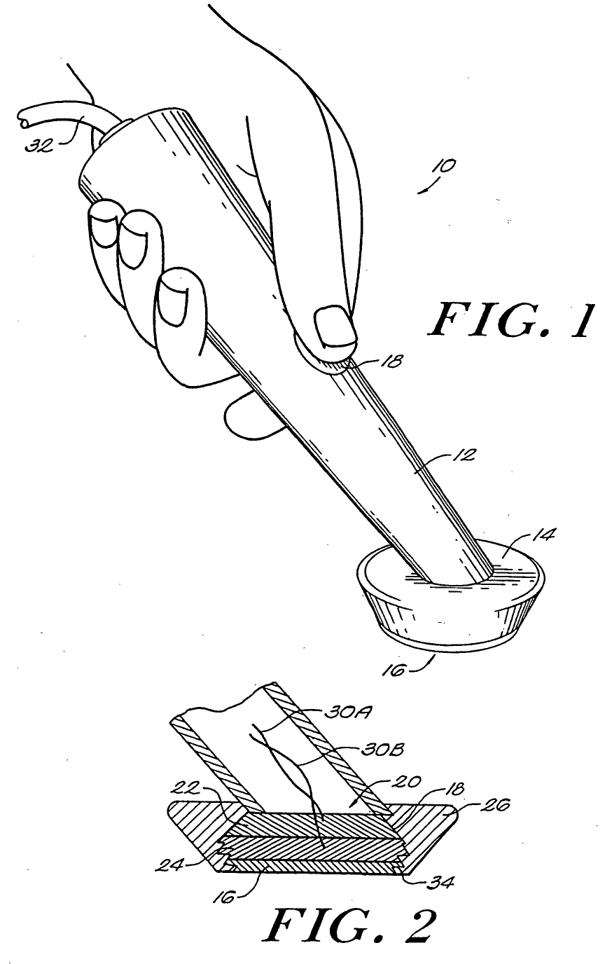

FIG. 1 illustrates a system 10 for ultrasound-mediated drug delivery, including a hand held probe 12, a transducer head assembly 14, a therapeutic agent dispenser 16, control switch 18 and an optional power cord 32 for connection to an external power source and/or auxiliary control circuitry.

In FIG. 2, a partially cross-sectional view of transducer head assembly 14 of FIG. 1 is shown including an ultrasound transducer 20 which can be formed by piezoelectric elements 22 and 24. Electrical lead wires 30A and 30B provide power and, optionally, a modulation or frequency control signals to the transducer elements. The electrical leads (or separate circuitry) can also carry feedback control signals picked-up by the transducers (in a monitoring mode) and convey such signals back to a controller (as discussed below). In addition, the body 26 of the transducer head can be constructed of a compliant polymeric material in order to conform to the tissue surface undergoing treatment, and can also include an internal cavity having walls 28 that reflect and/or focus the ultrasound energy to a desired site beneath the applicator. The transducer head assembly, in use, can also include a dosage dispenser 16 which applies the topical agent to the skin where transdermal penetration is desired. As shown in FIG. 2, the body of the transducer head assembly 26 can further include a snap-on coupler (e.g., a ring-like ridge) to facilitate coupling of the dosage dispenser 16 to the head assembly 14.

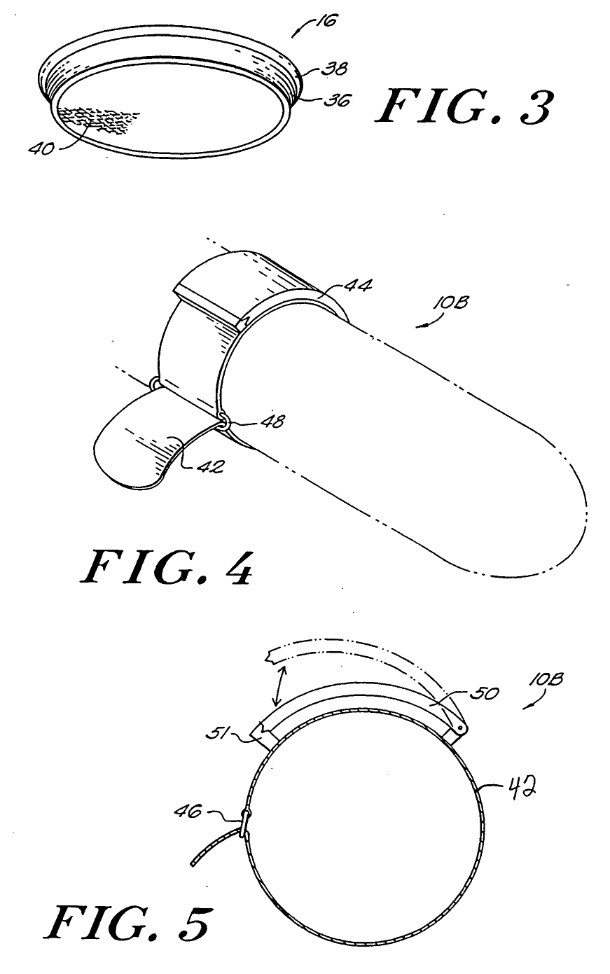

FIG. 3 provides a further illustration of a drug dosage dispenser 16 for use in conjunction with the transducer head assembly of FIG. 2. The dosage dispenser 16 includes a semi-rigid casement 36 having a rim or ridge coupler 38 which cooperates with the mating element 34 on the transducer head assembly to deploy and secure the dosage dispenser. The dosage dispenser 16 further includes a therapeutic agent 40, possibly together with additional physiologically-acceptable carriers, solvents or transdermal enhancing agents. The dispenser 16 of FIG. 3 can further include a peel off covering (not shown) to maintain sterility of the therapeutic agent until it is applied to the skin. The drug dispenser 16, if a snap-in coupling is employed, can also include one or more tab elements (not shown), to assist in detachment of the dispenser following the ultrasound-mediated therapy.

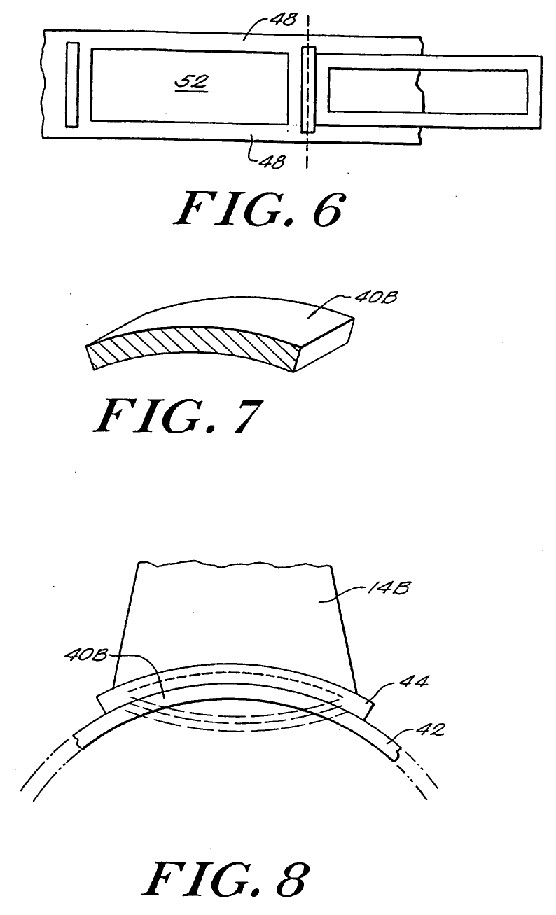

In FIG. 4 an alternative embodiment particularly useful in treating erectile dysfunction. The system 10B of FIGS. 4-8 includes a belt 42 which facilitates placement of the device around the penis of a subject. Connected to the belt 42 is a drug dispenser housing 44. The system can further include a cinch mechanism 46 to hold the system in contact with the subject's skin. Alternatively, the belt element can be a strap, optionally provided with an adjustment means, such as a slidable clasp, or other adjustable closure, for adjusting the fit of the device. Belt 42 can also be a band of an elastic material sized to provide a conforming fit for the user.

As shown in more detail in FIGS. 5, 6 and 7 the drug dispenser housing 44 can include walls 48 which define a receptacle 52 for a therapeutic agent 40B. The agent can be delivered as a gel or as a semi-solid block of medication. Alternatively, therapeutic agent 40B can be delivered in a dispenser casement similar to that described above in connection with the embodiment of FIGS. 1-3.

The apparatus 10B of FIGS. 4-7 is designed to cooperate with a ultrasound applicator as shown in FIG. 8. The applicator includes a transducer head assembly 14B which is sized and shaped to couple with the receptacle 52 or drug dispenser housing 44. When the therapeutic agent 40B is disposed within the receptacle, in contact with the skin, the ultrasound applicator and transducer head 14B can then be applied to agent 40B or housing 44 to effect transmission of ultrasonic waves (and penetration of the agent) through the skin as shown in FIG. 8.

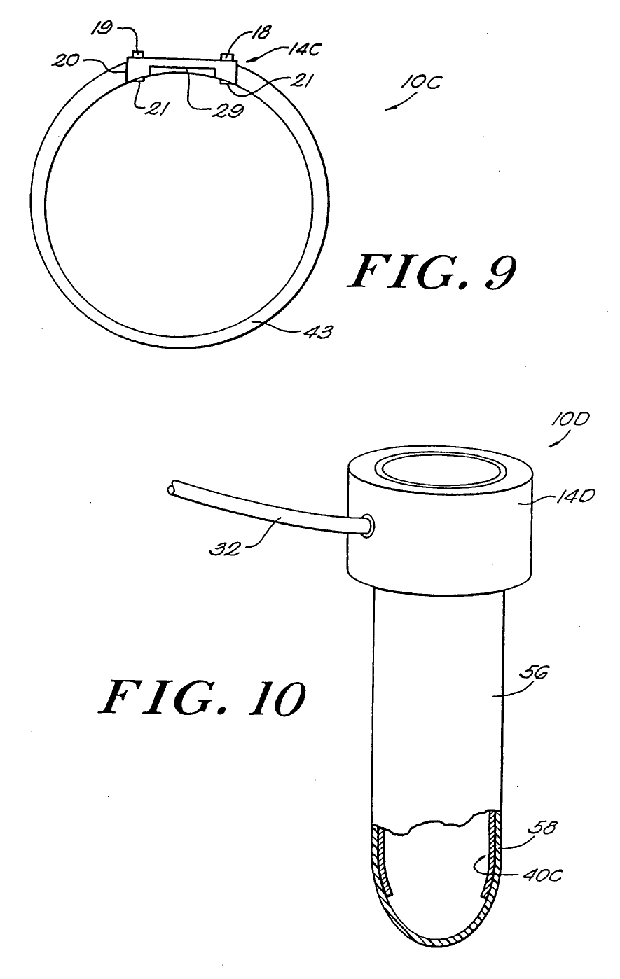

Another embodiment of a device of the invention is depicted in FIG. 9. As shown in FIG. 9, the device 10C can include an attachment means 43 for securing a miniaturized ultrasound source 14C to the penis of a user. Attachment means 43 can be a strap or an elastic band or belt similar to the belt and cinch mechanism described above in connection with the embodiment of FIGS. 4-8. Ultrasound source 14C includes a housing 27 for containing an ultrasound transducer, microelectronic circuitry suitable for controlling the operation of the ultrasound transducer, and, preferably, a power source such as a battery. The housing 27 is preferably constructed of a strong, durable, preferably compliant material; e.g., a plastic material such as a polyethylene, silicone or polyurethane composition, which advantageously also provides electrical insulation to protect the user against electrical shock.

In the embodiment of FIG. 9, housing 27 is provided with a slot 29 for receiving a source of the drug to be applied to the skin of the user. The drug composition, such as a gel or cream, can be dispensed by the user into the slot 29 before the device is used. In another embodiment, the drug is adsorbed or contained in unit dosage form as a patch, e.g., a transdermal patch, and the patch is sized to releasably and replaceably fit within slot 29. In such an embodiment, the patch is preferably designed for one-time use, with a suitable single does of the active agent, and then discarded after use. This embodiment provides a reliable metering of the drug and is convenient and simple to use while maintaining appropriate cleanliness of the device. The miniaturized device of FIG. 9 is preferably light weight and can, in one embodiment, present a rounded shape and low profile so as to not interfere with sexual intercourse. The device 10C can also be integrated into, or otherwise coupled to, a condom, if desired.

The embodiment of FIG. 9 depicts ultrasound source 14C with switch means 18 for controlling operation of the device, e.g., an on/off switch. The device can also be provided with one or more additional buttons 19 for allowing the user to adjust the operating parameters of the device (e.g., the frequency, duty cycle, or duration of the ultrasound energy application). The device 10C can further include one or more pressure transducers 21 to monitor the tumescence of the penis, thereby allowing discontinuance of the ultrasound energy when the penis has become erect (or reinitiation or the system in the event that the erection is lost).

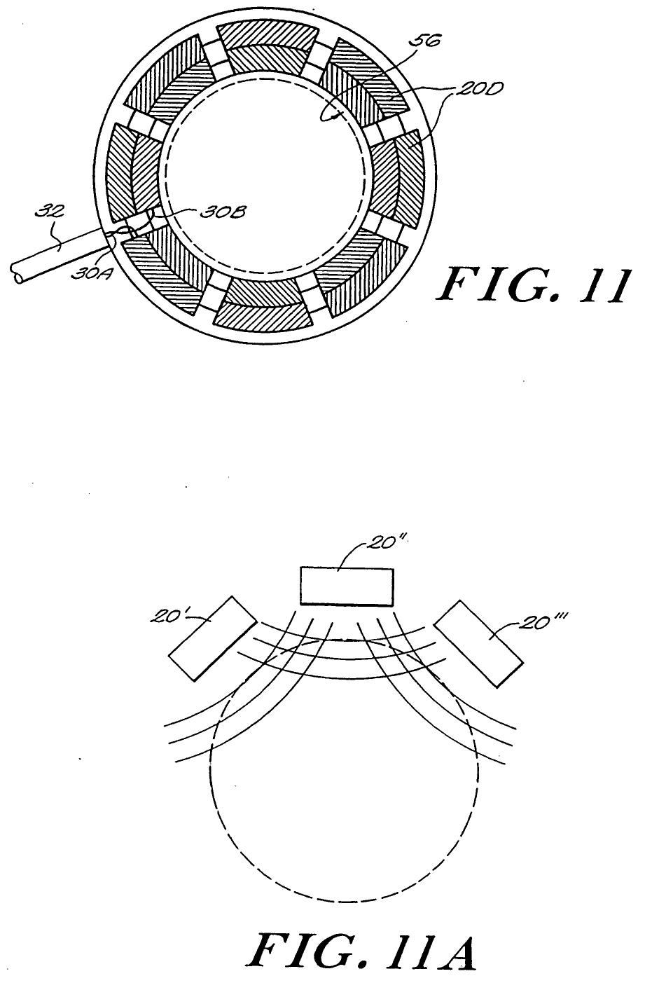

FIG. 10 illustrates yet another alternative system 10D for ultrasound-mediated drug delivery. The system 10D of FIG. 10 includes a toroidal transducer head assembly 14D having an hollow interior 60. The transducer assembly 14C can be activated by on-board batteries and/or a microprocessor element incorporated into the body of the transducer assembly 14D. Alternatively, an external power supply and/or external control circuitry can be applied to the device via electrical connection 32. As shown in more detail in FIGS. 10 and 11, the system 10C is designed to cooperate with a ring-like or condom-like sheath which is first applied to the penis. The sheath 56 includes a skin 58 and an internal coating of the therapeutic agent 40C. As illustrated in FIG. 10, the therapeutic agent can coat most or all of the sheath 56. Alternatively, the therapeutic agent can be confined to that portion of the sheath which is disposed between the transducer assembly 14 and the penis. In FIG. 11 a toroidal arrangement of transducer elements 20C is illustrated. These transducer elements can be coupled together by electrical leads 30A and 30B. Also shown in phantom in the cross-sectional illustration of FIG. 11 is the sheath element 56 having its internal drug coating.

FIG. 11A illustrates the particular advantage of employing a toroidal arrangement of transducers. FIG. 11 shows a partial toroidal arrangement of transducer elements 20′, 20″ and 20′″ and the interaction of their ultrasonic waves as they propagate into the stratum corneum. As the waves overlap each other, areas of constructive interference are created to locally increase the magnitude of the wave. This modulation is particularly useful in inducing temporary cavitation, one of the principal mechanisms for ultrasound-mediated transdermal penetration.

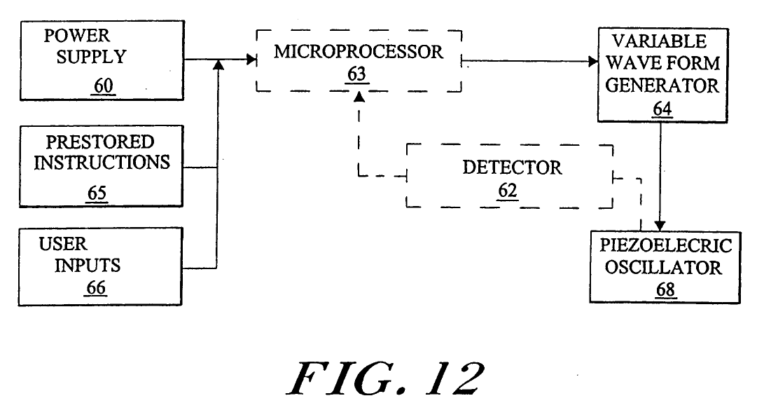

FIG. 12 illustrates a control system which can further ensure optimal ultrasound mediation. The system includes a power supply 60, a variable wave generator 64, stored instructions 65 or user input 66 and a piezoelectric oscillator 68. The system can further include a detector 62 and/or microprocessor 63 to monitor any feedback signals picked up the piezoelectric oscillator in a receiver-mode and/or provide control signals based on such feedback signals, prestored instructions or user inputs.

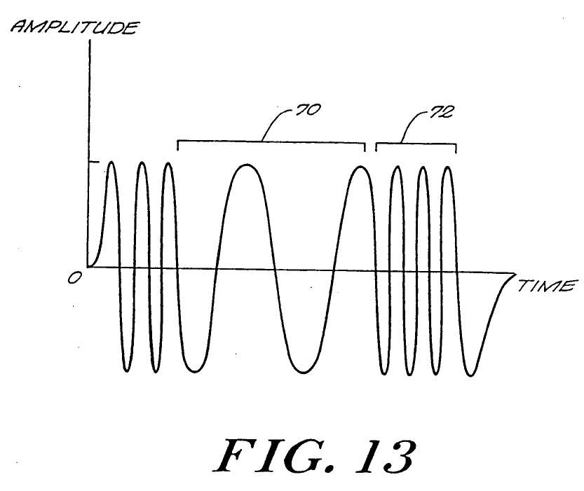

FIG. 13 illustrates the advantages of variable frequency ultrasound application. By modulating the frequency between a relatively longer wavelength region 70 and a shorter wavelength region 72, constructive interference can again be induced in the strata corneum, also enhancing cavitation.

The power, frequency, and duty cycle of the ultrasound energy should be selected to be sufficient to promote transdermal penetration of topical applied agents while substantially avoiding undesirable side effects such as heating or disruption of tissue. Thus, in certain embodiments, the ultrasound energy is applied in the frequency range of about 20 kHz to about 5 MHz, more preferably about 100 kHz to about 4 MHz, and still more preferably from about 500 k Hz to about 3 MHz, and preferably at a power of about 0.02 to about 3 watts/cm², more preferably about 0.2 to about 2 watts/cm², and still more preferably 0.5 to about 1.5 watts/cm². The ultrasound energy should be applied for a time sufficient to achieve the desired therapeutic result (i.e., treatment of erectile dysfunction) while avoiding tissue damage or discomfort to the subject. For treatment of erectile dysfunction, the time period for application should be relatively short. For other therapies, such a muscle sprains or hair loss therapy, the treatment can be longer. Exemplary time periods for application of ultrasound energy to the tissue of the subject can range from about 1 minute to about 2 hours, more preferably from about 2 minutes to about 1 hour, still more preferably about 5 minutes to about 30 minutes.

The duty cycle of the ultrasound source should preferably be from about 10% to about 60%, more preferably from about 20% to about 50%. It will be appreciated in light of the teaching herein that the power, duty cycle, and frequency of the ultrasound can be varied to achieve a desired therapeutic result without causing tissue damage such as burning. For example, ultrasound at a frequency of about 20 kHz can cause skin burns is applied at excessive power levels for extended periods of time. Thus, when a frequency of 20 kHz is employed, it is preferable to use a duty cycle and power level (e.g., 0.02 W/cm²) that will minimize tissue damage. For exemplary details on the construction and operation of ultrasonic applicators the teachings of U.S. Pat. No. 5,618,275 issued to Bock on Apr. 8, 1997; U.S. Pat. No. 5,421,275 issued to Lipkovker on Jun. 6, 1995; and/or U.S. Pat. No. 5,267,985 issued to Shimada et al. on Dec. 7, 1993 are incorporated herein by reference. One of ordinary skill in the art will be able to select appropriate power levels, duty cycles, and frequencies of ultrasound using no more than routine experimentation.

The invention has reduced to practice and the principles of the invention have been proven by animal experiments. In these experiments, male dogs, one year old, were anesthetized and a midline abdominal incision was made from the xyphoid process down to the symphisis pubis. Proceeding slightly lateral to the left side of the penis, the corpus cavemosum was exposed. Two butterfly needles were placed into one of the corpus cavemosum, one proximally and one distally. The proximal needle was used for intracorporal pressure recordings, while saline was infused through the distal needle. Controlled intracorporal blood pressure (without applying any drugs) was monitored and recorded while saline was perfused at a rate of 1.7 ml/min. Nude mice skin was used to cover the exposed corpora by stitching it to the dog's incised skin. A gel containing 500 mg papaverine (in a base of polyethylene glycol, methyl paraben, butyl paraben, and butylated hydroxytoluene) was applied on the surface of the nude mice skin followed by the application of an ultrasound probe of 1 MHz, pulse mode 30%, at a power level of 2 w/cm², for 20 min. In the control experiments, papaverine was applied topically without any ultrasound application and the manometric pressure was monitored. The resulting manometric pressure was compared to the control pressure by measuring the time needed to reach the peak pressure and the time needed for the cavernosal pressure to decline to baseline.

Perfusion of the corpora with saline (with no ultrasound or papaverine application) was performed for 15 sec at a rate of 1.7 ml/min. creating a pressure ranging from 150-220 cm. H₂0. The time needed for this pressure to drop to baseline (0-5) was 30 sec. Perfusion of the corpora with saline after applying ultrasound and papaverine reached the same pressure peak in only 5 sec. Moreover, the pressure declined to baseline in 1.5 minutes. Applying only papaverine (no ultrasound) on the skin for 20 min. gave the same results as in the control experiments, where only saline was perfused.

The experimental results demonstrate an absence of topical effect for papaverine alone; that is, papaverine gel, without application of ultrasound, resulted in no greater pressure readings than the no-drug control. These findings are consistent with the human clinical experience, suggesting that penetration of the drug from the skin surface to the corpus cavemosum is insufficient to provide effective therapy in the absence of an enhancer of drug penetration or absorption. However, the addition of the ultrasound treatment was able to create a rise in pressure which was consistent with erection. Therefore, these results indicate that ultrasound enhanced the permeability of papaverine into the corpus cavemosum tissue, increasing arterial inflow and affecting the resistance to venous outflow. The effects of papaverine were achieved with a 300% improvement in the period of time established as a baseline for the establishment of erection. Furthermore, the effects were 500% more persistent than with the baseline erection parameters.

The contents of all references, patents, and published patent applications cited throughout this application, including the background, are hereby incorporated by reference.

Those skilled in the art will recognize, or be able to ascertain using no more than routine experimentation, numerous equivalents to the specific procedures described herein. Such equivalents are considered to be within the scope of this invention and are covered by the following claims.

Other embodiments are within the following claims.

1-50. (canceled)

51. A device for administration of topical therapeutic agents, comprising an applicator for applying an effective amount of a therapeutic agent to a tissue surface of a subject; an ultrasound transducer, operatively coupled to the applicator, for providing ultrasound energy to the tissue surface at least one predetermined frequency to promote absorption of the agent through the tissue of the subject, and a pressure-sensitive switch to determine an erectile state of a penis, wherein the ultrasound energy has a duty cycle greater than about 10%, and wherein the ultrasound transducer further comprises at least one oscillating element capable of generating ultrasound energy at a frequency of greater than about 500 kHz and less than or equal to about 3 MHz, whereby the frequency promotes the therapeutic effect of the therapeutic agent.

52. The device of claim 51, wherein the ultrasound transducer further comprises at least one oscillating element capable of generating ultrasound energy at a power of about 0.02 to about 3 watts/cm².

53. The device of claim 51, further comprising a controller for varying the frequency of the ultrasound energy.

54. The device of claim 51, further comprising a controller for varying the power of the ultrasound energy.

55. The device of claim 51, further comprising a compliant skin contacting material.

56. The device of claim 51, further comprising a detector for monitoring feedback signals from the transducer.

57. The device of claim 51, wherein the tissue is a penile tissue and wherein the therapeutic agent is selected from the group consisting of papaverine, prostaglandin E1 (PGE 1), prostaglandin E2 (PGE 2), prostaglandins, organic nitrites, inhibitors of the renin-angiotensin system, inducible Nitric Oxide Synthase (iNOS) agents, enzyme inhibitors, vasoactive agents, phosphodiesterase inhibitors, and smooth muscle relaxant.

58. The device of claim 51, wherein the phosphodiesterase inhibitors are phosphodiesterase type 5 inhibitors selected from the group consisting of sildenafil and alprostadil.
